(19) United States
(12) Patent Application Publication
(10) Pub. No.: US 2011/0223589 A1
(43) Pub. Date: Sep. 15, 2011

(54) Solid Phase Nucleic Acid Extraction From Leukoreduced Blood

(75) Inventor: Chiu Chau, Edison, NJ (US)

(73) Assignee: BIOSAMPLE LLC, Edison, NJ (US)

(21) Appl. No.: 13/019,511

(22) Filed: Feb. 2, 2011

Related U.S. Application Data
(60) Provisional application No. 61/311,825, filed on Mar. 9, 2010.
(60) Provisional application No. 61/407,197, filed on Oct. 27, 2010.
(60) Provisional application No. 61/410,045, filed on Nov. 4, 2010.

(51) Int. Cl. C12Q 1/68

(52) U.S. Cl. 435/6.1

(57) ABSTRACT

Products for and a method of capturing and storing nucleic acid from leukoreduced patient blood, where the products capture sufficient quantities for use in PCR and analysis of nucleic acid, are described. The products are made by heating a mixture of magnetic beads, silica particles, alkyl silicate (e.g., Silbond 4, Silbond Corp., Weston, Mich.) and polyethylene resin particles. The quantity of nucleic acid adsorbed by the product is controlled by the surface area of silica available for binding to nucleic acid, which in turn is controlled by: the overall volume of the product, the ratio of the volume of polyethylene resin particles to the volumes of silica particles and alkyl silicate; and the sizes of silica particles (smaller particles have a larger surface area per unit volume).

### RELATED APPLICATIONS

This application claims priority to U.S. provisional Nos. 61,311,825, filed Mar. 9, 2010; 61,407,197, filed Oct. 27, 2010; and 61,410,045, filed Nov. 4, 2010.

### FIELD OF THE INVENTION

The invention relates to solid phase capturing and storing of DNA or RNA from a small volume of leukoreduced blood, and releasing a controlled quantity of DNA or RNA from the solid phase.

### BACKGROUND

Whole blood is generally the least expensive and most readily accessible source for genomic DNA. It has the further advantage of providing immediate visual evidence that a sample of adequate size has been obtained. However, isolating DNA from fresh or frozen blood is difficult, since only 0.1% of blood cells are nucleated white blood cells (10×10⁷/ml)—red blood cells have no nucleus or DNA. For example, 1 μl of lysed human blood contains only about 35-50 ng DNA amid about 150 μg of protein, lipids and other components.

A variety of techniques and devices have been developed to isolate DNA from blood. For example, in the most rigorous protocols, several milliliters of whole blood are drawn and then centrifuged to separate blood into: plasma; a white blood cell (WBC) rich fraction (buffy-coat); and, a red blood cell (RBC) rich fraction. The WBC's are first isolated and the DNA is released using detergent lysis, followed by protease treatment and DNA purification using phenol-chloroform extraction, followed by ethanol or isopropanol precipitation of the DNA (Sambrook, J. et al. 1989. Molecular cloning, 2^{nd} Ed Cold Spring Harbor Laboratory Press, Cold Spring Harbor, N.Y.). The simplest reported method of DNA extraction involves boiling 1-3 μl of blood in 50 μl of water for cell lysis, and directly using a portion of the lysate for further analysis (Skalnik, D. G. and Orkin, S. Biotechniques 8: 34 (1990)).

The most widely-used and versatile among the currently available methods of DNA extraction are the solid phase based separation methods. In these methods whole blood is lysed in the presence of an appropriate buffer that allows the released DNA to selectively adsorb onto a solid phase. This is followed by a wash step which selectively washes away the non-specifically adsorbed components, leaving the adsorbed DNA. Finally the adsorbed DNA is eluted using an appropriate elution buffer. Several solid phase DNA extraction methods have been discussed, including:

U.S. Pat. No. 6,043,354 (to Hilebrand et al.) discusses simultaneous two step extraction of DNA and RNA using a solid phase/buffer combinations.

U.S. Pat. No. 5,523,231 (to Reeve et al.) discusses a method of macromolecule (DNA) recovery from a solution containing magnetic beads via induced macromolecule precipitation which generates macromolecule-magnetic bead aggregates. The aggregates are then separated and the DNA recovered.

U.S. Pat. No. 5,898,071 (to Hawkins et al.) discusses a method for reversibly and non-specifically binding polynucleotides to a functionalized solid using a combination of chaotropic salt and buffer, followed by recovery of the bound DNA.

U.S. Pat. No. 7,173,125 (to Deggeradal et al.) discusses isolation of nucleic acids from a sample using detergents and magnetic beads.

U.S. Pat. No. 5,234,809 (to Boom et al.) discusses a method for isolating nucleic acids from complex nucleic acid-containing starting materials (blood, serum etc.) in a one step method using silica particles and chaotropic salt.

U.S. Pat. No. 5,582,988 (to Backus et al.) discusses a method for selective nucleic acid capture and release using weakly basic polymers and different pH.

U.S. Pat. No. 5,945,525 (to Uematsu et al.) discusses a method for nucleic acid separation using silica coated super-paramagnetic particles.

U.S. Pat. No. 6,027,945 (to Smith et al.) discusses a method for nucleic acid separation using silica coated magnetic particles.

No matter the nature of the protocol, the recovery efficiency and the final yield of the DNA is critically dependent on having sufficient numbers of nucleated cells in the initial blood sample. None of the prior art methods address the problem of extracting sufficient DNA from leukodepleted blood samples.

Leukodepletion is a process by which leukocytes (WBCs) are removed from donated blood. It is well established that a majority of febrile nonhemolytic adverse transfusion reactions are mediated by donor leukocytes. The use of leukoreduced products is thus indicated for multi-transfused patients, patients receiving chemotherapy, patients undergoing bone marrow, renal or peripheral blood progenitor cell transplant, and patients with hematologic malignancies. Current standards also require that, as a minimum, blood selected for transfusion to a patient be checked (phenotyped) to be antigen negative to the existing alioantibodies in the patient's serum. Recently DNA analysis has emerged as a powerful, versatile and cost effective method for blood group antigen phenotype determination (Hashmi, G. et al. Transfusion, 45, May 2005; 680-688; Hashmi, G. et al. Transfusion, 47, April 2007, 736-747). DNA analysis using blood relies on the fact that white blood cells (WBCs) are the only cells in blood carrying genomic DNA.

When the starting WBC concentration in whole blood is very low (as in the case of leukodepleted samples), it is difficult to carry out DNA based assays using standard DNA extraction protocols. Highly sensitive quantitative PCR techniques have been utilized to characterize the extracted DNA from leukoreduced blood samples (Lee, T.-H. et al. Transfusion, 42 (1) 87-93 (2002)). However, such assay techniques require sophisticated technicians, use dedicated and expensive instrumentation and typically cannot be multiplexed. There is no currently established method or commercially available kit that can utilize leukodepleted blood as a source of genomic DNA for highly multiplexed genotyping assays—which are becoming the standard for blood group antigen phenotype determination.

U.S. Pat. No. 6,670,128 B2 (to Smith, et al.) discusses a method for utilizing spent leukodepletion filter devices as a source material for the isolation and analysis of genomic DNA.

However, leukodepletion is routinely carried out only in larger blood centers and hence such leukocyte loaded filter devices are only available at a limited number of facilities. In most places, leukodepleted donated blood is available stored in a soft plastic blood collection bags. Because of potential contamination of the blood that may occur from contact with a syringe or pipette used to withdraw a sample, the blood collection bag is connected to a flexible plastic tube that is heat sealed into a series of segments containing the donor's blood. These sealed tube segments are commonly referred to as segment tubes, pigtails, or segments. The segment tubes remain attached to the blood collection bag, and are often folded into a group held together with a rubber band. Whenever the blood is to be tested, the laboratory technician simply removes one or more of the segment tubes attached to the blood collection bag for testing. Since the volume of leukodepleted blood available from the segments is limited, such segment samples cannot be utilized for extracting genomic DNA using the filtration device based recovery process as described in U.S. Pat. No. 6,670,128. It has been estimated that the average content of WBCs in donated human whole blood is 10⁹/unit. When leukodepeletion is by the currently used methods, the total content of WBCs in a leukodepleted blood unit would be less than 5×10⁶/unit or about 10 WBC's/μl. While it is intuitively clear that theoretically, starting with a 3 log higher volume of blood one can compensate for the leukodepletion, such volumes are impractical to obtain. What is required then is a pre-concentration step for the leukocytes from the leukodepleted blood.

Various approaches which allow selective separation and subsequent analysis of WBCs from whole blood are known.

U.S. Pat. No. 5,155,044 (to Ledis et al.) discusses method and reagent system for the rapid isolation, identification and/or analysis of leukocytes from whole blood sample.

U.S. Pat. No. 6,869,798 B2 (to Crews et al) discusses a lytic reagent composition and the method of its use for differential analysis of leukocytes using flow-cytometry.

U.S. Pat. No. 5,789,147 (to Rubenstein et al.) discusses a method for separating high concentrations of WBC's having a high degree of cell viability via low speed centrifugation of blood bags.

U.S. Pat. No. 5,155,044 (to Veriac et al.) discusses a lytic reagent composition for simultaneous measurement of hemoglobin and determination of leukocytes in blood sample comprising a cationic detergent, a compound of the glycoside type and at least one inorganic salt and/or an osmotic and/or leuko-protective agent.

All of the methods described above, though capable of efficient pre-analytic lysing of RBCs to allow analysis of WBCs using flow cytometry or otherwise, are not suitable for WBC pre-concentration method from leukoreduced samples. For example, successful implementation of Veriac et al.'s method (U.S. Pat. No. 5,789,147) requires a high starting blood sample volume, which is not practical when analyzing leukoreduced segment samples. Use of protocols outlined in U.S. Pat. No. 5,155,044, U.S. Pat. No. 6,869,798 or U.S. Pat. No. 5,155,044 lead to an undesirable high dilution of the recovered intact WBC's, which necessitates further use of lengthy and repeated procedures for re-concentrating the WBC's in a small volume (less than 1 ml), to make the sample suitable for use in standard solid phase DNA extraction procedures.

Various microfluidic methods have also been described for separation of leukocytes from whole blood (see Sethu et al. Lab Chip, 2006, 6, 83-89, Shevkoplyas et al., Anal. Chem. 2005, 77, 933-937). Such methods, though promising, are not very efficient or easy to use.

WBCs can also be separated from blood using commercially available lymphocyte separation medium (Ficoll-Paque PLUS, GE Healthcare, Piscataway, N.J.). The method involves layering a given volume of whole blood on top of the Ficoll-Paque Plus media and subjecting the mixture to a short, low speed centrifugation. The erythrocytes and the granulocytes sediment to the bottom of the tube and because of their lower density, the lymphocytes are collected at the interface between the plasma and Ficoll-Paque Plus media. Though this method can be adapted to small volumes of blood, the method is time consuming and rather inefficient in terms of WBC recovery yields (typically less than 30%) when using small volumes (<1 ml).

At present, therefore, no method can rapidly and effectively perform DNA extraction from small volumes of leukodepleted blood samples and give a reasonably high extraction yield.

### SUMMARY

A process of extraction and analysis of patient nucleic acid (DNA or RNA) from leukoreduced blood samples is described. Products for capturing nucleic acid from leukoreduced patient blood are made by heating a mixture of magnetic beads, silica particles, alkyl silicate (e.g., Silbond 4, Silbond Corp., Weston Mich.; ethyl polysilicates) and polyethylene resin particles so as to weld the polyethylene resin particles together with silica particles and alkyl silicate, with the magnetic beads embedded in the melt, such that, as a result of the melting/welding of the polyethylene resin particles, fluid pathways are formed between the welded particles. If the product is immersed in leukoreduced patient blood, or if leukoreduced blood is flowed through the product, nucleic acid in the sample will be absorbed by the silica surfaces (formed by the silica particles and alkyl silicate), which line the outer surfaces of the product and the insides of the fluid pathways.

The amount of nucleic acid adsorbed by the product is controlled by the surface area of silica available for binding to nucleic acid, which in turn is controlled by: the overall volume of the product, the ratio of the volume of polyethylene resin particles to the volumes of silica particles and alkyl silicate; and the sizes of silica particles (smaller particles have a larger surface area per unit volume). Controlling and limiting of the amount of nucleic acid captured by the product avoids the disadvantages associated with excess DNA/RNA for PCR amplification.

The proportion of magnetic beads will generally not affect nucleic acid adsorption because they are smaller in size and present in small amounts—and thus, have small volume in the product as compared with the resin particle and total silica volumes. The magnetic beads permit the product to be magnetically attracted allowing easier, or automated, movement.

The volumes of polyethylene resin particles silica particles in the product relate to the distribution of sizes of the particles. That is, the particle size ranges can be determined, and the numbers of each type of particles of a particular size controls the volume of that particle in the product. The numbers of each particle type can be averaged and standard distributions determined.

The product also allows extraction of DNA/RNA from limited volumes or numbers of cells. In the case of extraction from stem cells or cancer cell, few cells are generally available. The invention can efficiently extract DNA in sufficient quantities for PCR amplification, from as little as 5 μl of solution or as few as 1-100 cells.

The selection of polyethylene resin particles of a certain size range (e.g., 8-300 μm) also allows the product to function as a filter, allowing preferential selection of DNA material of certain sizes. For instance, one could select the smaller mitochondrial DNA by reducing the size of the resin particles so that the larger DNA cannot pass through the pathways, and only the smaller size DNA is captured.

It is an advantage to have several final products bearing the same patient nucleic acid, as they can be separately analyzed, or stored for later analysis. As DNA analysis for disease diagnosis and treatment, identification, genetic cross-matching and other purposes increases, a system for long term nucleic acid retention is increasingly useful. To identify several final products with one patient, the product can be bar-coded, or identified with another coded identification system (including RFID or nucleic acid tags), so that patient confidentiality can be maintained.

In preferred operation, the marked products are placed into a container and a patient's blood or fluid sample is added to the container, whereby nucleic acid in the blood or fluid sample is adsorbed by the silica surfaces. After adsorption, the products are removed from the container, preferably using a magnetic device, which attracts the magnetic beads to pull them from the container.

When marked/identified products are used, the patient's nucleic acid is not separated from the product, by elution or otherwise, before the nucleic acid is amplified. The marked products are placed in a container for amplification and the amplification is carried out on the solid phase product. The amplified nucleic acids in the container can be analyzed in situ, or the fluid in the container can be removed and they can be analyzed separately. The marked product is stable and can be stored for later use and analysis.

Because elution of nucleic acid from the product is eliminated as a step in the process, the process can also be readily automated. A robot simply uses magnetic attraction to lift the product from the sample container, and it is then placed in the container where PCR or other amplification, and analysis, takes place. Alternatively, an operator can perform this step.

### BRIEF DESCRIPTION OF THE DRAWINGS

FIG. 1 is depicts the product for nucleic acid extraction and storage.

FIG. 2 schematically depicts the components of the product of FIG. 1 in a mold inside an oven.

FIG. 3 shows the work-flow for the capture of nucleic acid from a patient sample with the product of FIGS. 1 and 2.

FIG. 4 shows the work-flow for nucleic acid extraction from a leukorduced sample—having few cells.

FIG. 5 shows real time PCR results, illustrating DNA extracted from four different leukoreduced blood samples and controls.

### DETAILED DESCRIPTION

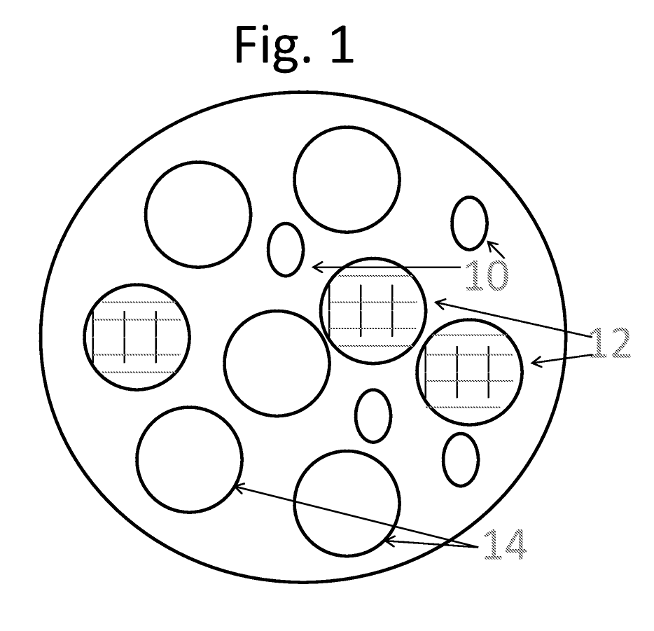

FIG. 1 is a plan view of the product for nucleic acid extraction. It includes magnetic beads 10, silica particles 12, alkyl silicate (not shown) and polyethylene resin particles (not shown) heated to weld the polyethylene resin particles together, thereby forming fluid channels (depicted as holes 14 in FIG. 1) through the product. Silica particles 12 are preferably about 5-20 μm in diameter. Polyethylene resin particles range from 8-1000 μm in diameter. Magnetic beads can be smaller, preferably from 0.1 to 2 μm in diameter. Controlling the relative proportion and size range of the polyethylene resin particles used in the product controls the size, the distribution of sizes and the number of fluid channels in the product, and the quantity of nucleic acid it can adsorb and ultimately release.

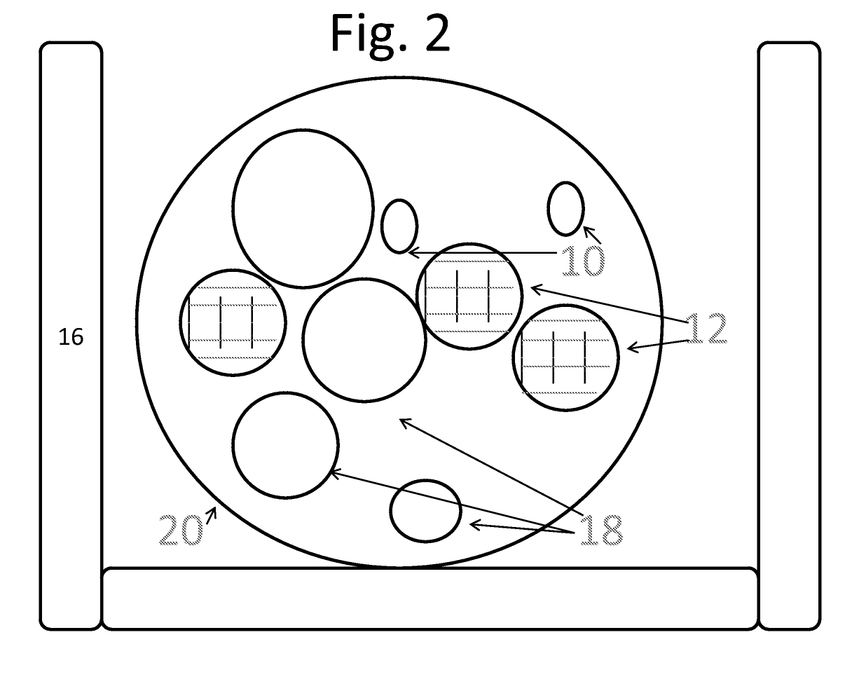

FIG. 2 depicts making the product, i.e., mix the ingredients (silica particles 12, alkyl silicate, not shown, and resin particles 18) in a mold 20 to form a three-dimensional structure (a disc in this case, but other shapes can be used), and place the mold 20 inside oven 16. Preferably, the oven is heated to 200° C., cooled to room temperature and release from the mold, then heated to 500° C. for one hour to finish the particle welding process.

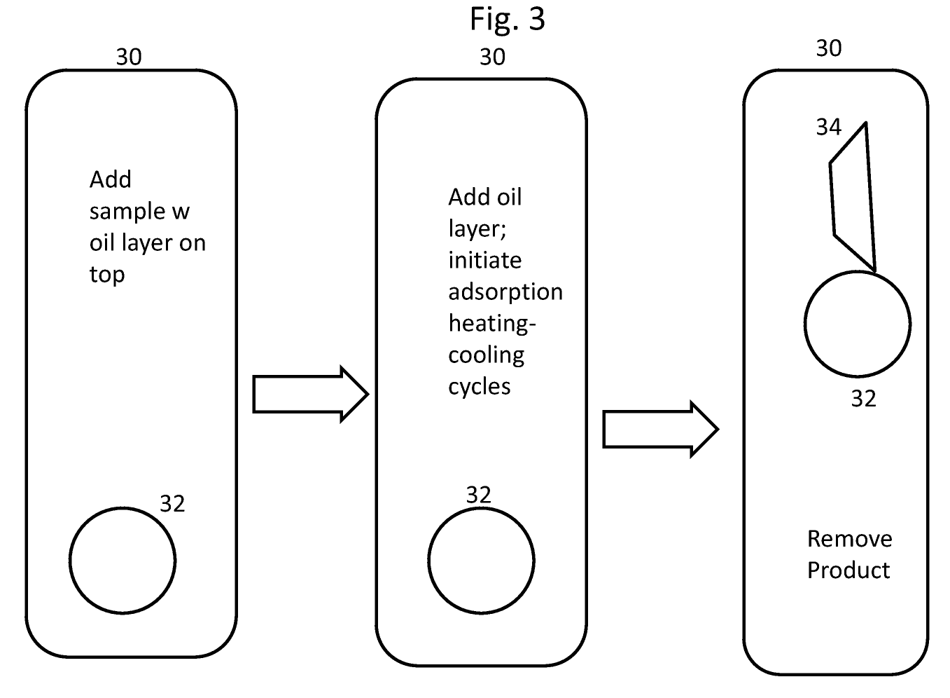

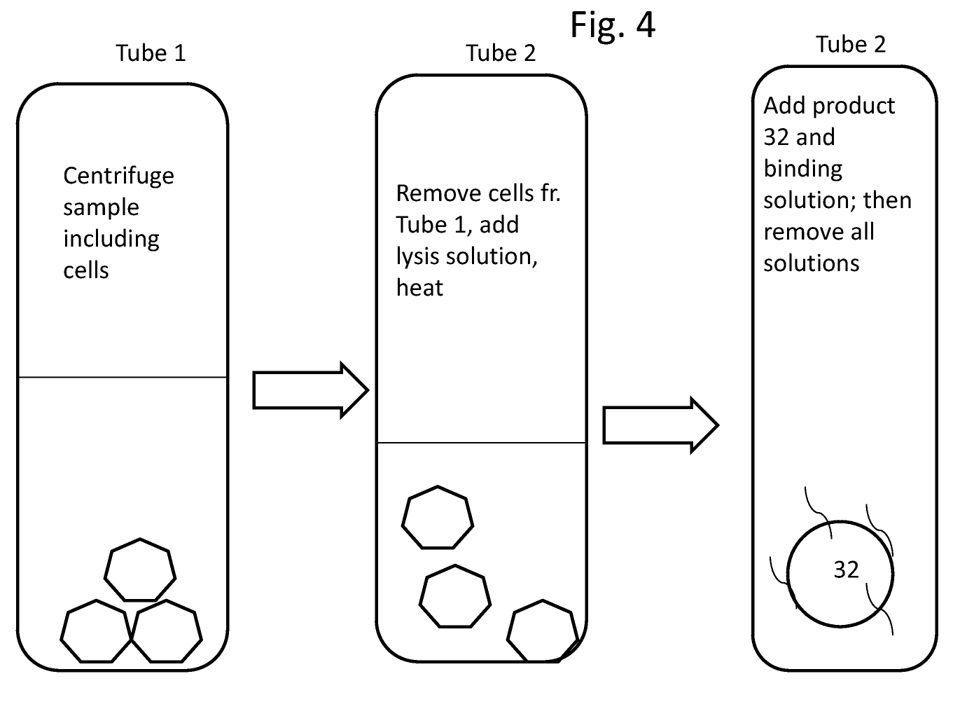

FIG. 3 depicts adsorbing DNA from a sample with a product 32 in the lower part of a tube 30, and FIG. 4 depicts adsorbing DNA from a leukoreduced sample. The product 32 is preferably coded using, e.g., a bar code, to identify the patient source. In FIG. 3, the sample with an oil layer on top, is transferred to the tube 30. Tube 30 can contain one or more of the products 32, which are preferably encoded e.g., with a bar code. Alternatively, the encoding can be by nucleic acid tagging or by comparing the unique patterns on each product (which are formed in its making). These patterns are stored as images which can be decoded later by taking another image and comparing, in order to identify any particular product.

The tube 30 also contains all the reagents needed for adsorption of DNA or RNA from the sample. Further oil is added on the upper surface of the sample, to protect the sample from airborne particles and contamination, and to isolate the sample (potentially a biohazard) from the work area and inhibit evaporation.

The sample is then put through a heating and cooling cycle—a typical cycle would be room temperature to 45° C. to 85° C. for 1-10 min, then RT-60° C. for 1-10 min to adhere nucleic acid to the product. The heating/cooling cycle runs preferentially at 65-81° C., then between RT and 48° C. for 5-20 cycles. Once the cycle finish, washing reagents (typically at 1-1000 μl) would be added to the tube to dilute the sample. Or the sample can be taken to a washing station for washing

A magnetic device 34 can be used to pick the product out of the tube (by attraction to the magnetic beads) and transfer it to a washing station for more extensive washing. Picking with device 34 can be part of an automated system—a robot can be controlling it, and initiating this action at the appropriate time. Also, the robot could place the product in tube 30 initially, then transfer at the appropriate time.

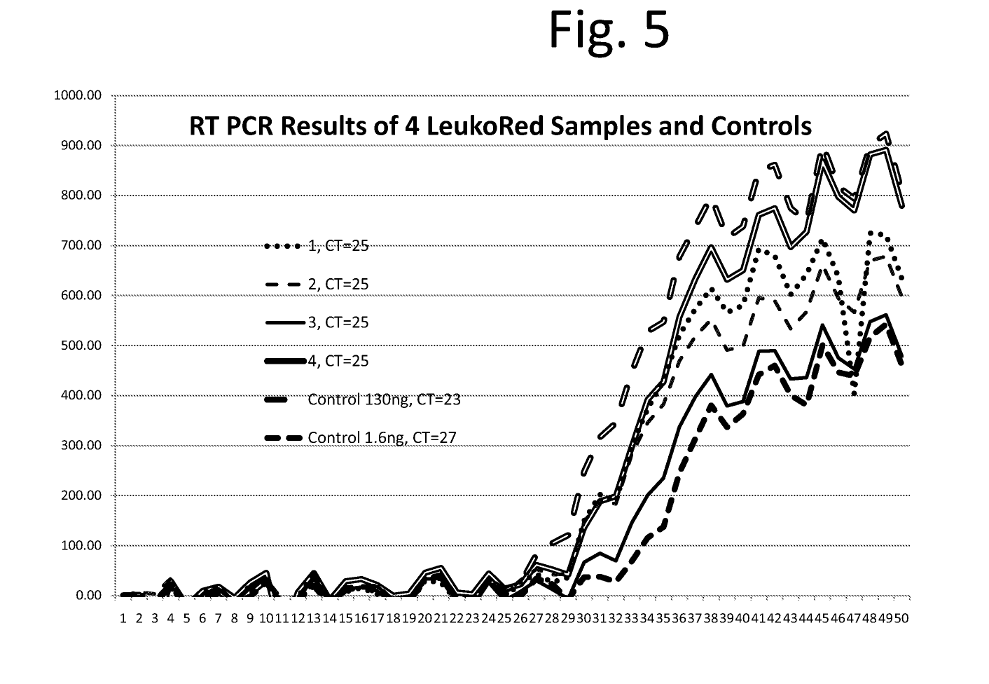

FIG. 5 show the results where DNA was extracted with a product as described herein, with the process above, from 50 μl of a leukoreduced blood sample. The product with the DNA adsorbed is amplified, and the signals are measured using Real Time PCR. The results demonstrate that sufficient DNA was extracted to run the real time PCR, and that the quantity of DNA extracted in this example was between 1.6 and 130 ng.

To determine the maximum amount a product as described herein can capture, 20 μl of a 1000 ng/ul DNA solution is used. The exemplary product for extraction is 1×1×1.5 mm and is incubated for 5 minutes after sample contact, and eluted with 10 μl water. As shown in Table I, by changing the proportion of resin in the product (where the resin particles are a particular size range and size range distribution) the amount of DNA released varies in accordance with the change in adsorbing surface area of the product. The amount of DNA adsorbed and released by the product will change with changes in the proportion of resin and the size range and size range distribution of the polyethylene resin particles, as explained above. In Table 1, the particle size ranges from 8-1000 μm in diameter, as determined by observation of a population of the particles. Populations of particles with size ranges of 8-1000 μm in diameter (assuming a similar size distribution in that range) when used in the same proportions as in Table I in making the product, would form products which would be expected to release the same amounts of DNA as indicated in Table I. If the distribution of sizes of the polyethylene resin particles shifts towards larger particles, the distribution of the fluid channel diameters will also increase, and the product will release more DNA. Similarly, shifting the distribution of particle sizes to smaller particles will result in a decrease in the amount of DNA the product releases.

Table 1 shows the experimental results where the proportion of polyethylene resin particles in the product (by volume) increases from 0 to 35% (particle size varies from 8-1000 μm). The product captures from 200 ng of DNA (where no resin was in product), 300 ng at 15%, 400 ng at 25% and 500 ng of DNA where resin is 35% by volume of the product. The experimental conditions were: for each run of product with a different proportion of resin: 10 μl of 1000 ng/μl pure DNA was contacted with two products as described herein (each was 1×1×1.5 mm, and with the proportion of resin and particle sizes indicated in Table I) in binding solution. This was followed by incubation at 75° C. for 5 minutes, and elution at room temperature with water for 5 minutes.

TABLE I

| Resin % by volume | ng of DNA Captured |
|---|---|
| 0 | 200 |
| 15 | 300 |
| 25 | 400 |
| 35 | 500 |

The ability to adjust the amount of DNA released is highly desirable for downstream PCR. The product described herein allows the amount of DNA released to be adjusted for optimal PCR.

It should be understood that the terms and expressions used herein are exemplary only and not limiting, and that the scope of the invention is defined only in the claims which follow.

What is claimed is:

1. A process for capturing sufficient quantities of patient nucleic acid from leukoreduced blood samples to permit amplification and analysis of said patient nucleic acid, comprising:
making products for capturing nucleic acid from patient samples, wherein individual products are made by heating a mixture of magnetic beads, silica particles, alkyl silicate and polyethylene resin particles so as to weld the polyethylene resin particles together, and such that fluid pathways are formed through the products; and wherein:
the surface area of silica available for binding said patient nucleic acid is adjusted to a desirable range for capturing sufficient quantities of patient nucleic acid by controlling: (i) the overall volume of the product; (ii) the ratio of the volume of polyethylene resin particles to the volumes of silica particles and alkyl silicate; and (iii) the sizes of silica particles.

2. The process of claim 1 wherein the alkyl silicate includes ethyl polysilicates.

3. The process of claim 1 wherein the patient nucleic acid is DNA or RNA.

4. The process of claim 1 wherein the proportion of polyethylene resin particles in the product varies from 0 to 35% by volume.

5. The process of claim 1 wherein the size range of the polyethylene resin particles is from 8-1000 μm in diameter.

6. The process of claim 1 wherein the size range of the polyethylene resin particles is from 8-200 μm in diameter.

7. A process of claim 1 wherein the samples are 100 or fewer patient cells.

8. The process of claim 1 further including the step of determining the distribution of the size range of polyethylene resin particles or silica particles.

9. The process of claim 8 wherein the distribution is defined as a specified proportion of the particles being within a specified size range or of a specified size.

10. The process of claim 8 wherein the standard deviation of the particles within the size range is determined.
